(19) United States
(12) Patent Application Publication
(10) Pub. No.: US 2014/0042081 A1
(43) Pub. Date: Feb. 13, 2014

(54) Filter for Removing Substances from Blood or from Blood Derivatives, and a Method for Obtaining It

(71) Applicant: GVS S.P.A, Zola Predosa (Bologna) (IT)

(72) Inventors: Massimo SCAGLIARINI, Casalecchio Di Reno (Bologna) (IT); Luca QUERZE, S. Lazzaro Di Savena (Bologna) (IT)

(73) Assignee: GVS S.P.A., Zola Predosa (Bologna) (IT)

(21) Appl. No.: 13/759,563

(22) Filed: Feb. 5, 2013

Related U.S. Application Data
(63) Continuation-in-part of application No. PCT/IB2011/001792, filed on Jul. 29, 2011.

(30) Foreign Application Priority Data
Aug. 6, 2010 (IT) MI2010A001514

(51) Int. Cl. A61M 1/36; B05D 3/02; B05D 5/00; B05D 1/18

(52) U.S. Cl. CPC A61M 1/3633; B05D 1/18; B05D 3/0209; B05D 5/00
USPC 210/496; 427/439; 427/382; 427/244; 427/354

(57) ABSTRACT

A filter for removing substances, including leukocytes and platelets by way of non-limiting example, from whole blood or from blood derivatives, the filter includes a casing containing a layered filter element, at least one layer of this latter being coated with polyurethane; this polyurethane has a number average molecular weight between 10,000 and 20,000 Dalton. A method for forming the layered filter element for a filter such as mentioned above, in which at least one layer of this filter element is impregnated with polyurethane by immersing it in a container of a mixture in which the polyurethane is present. This mixture includes a solution in which the polyurethane is dissolved in a polar solvent such as water.

### CROSS-REFERENCE TO RELATED APPLICATION

This is a continuation in part of Patent Cooperation Treaty application no. PCT/IB2011/001792 filed 29 Jul. 2011 which claims the benefit of priority from Italian patent application no. MI2010A001514 filed 6 Aug., 2010, all incorporated herein by reference in their entirety.

### FIELD OF THE INVENTION

The present invention relates to a filter for removing substances, in particular such as leukocytes and platelets, from blood or from blood derivatives, in accordance with the introduction to the main claim. The invention also relates to a method for forming the aforesaid filter, in accordance with the corresponding independent claim.

### BACKGROUND OF THE INVENTION

Filtration of blood components for transfusion is a known practice usually performed on the blood withdrawn from a donor. The main purpose of this filtration or purification is to reduce as much as possible the presence of leukocytes in the transfused blood (leukodepletion) and the presence of platelets therein in order to reduce possible deleterious effects related to transfusion, such as: alloimmunization; non-hemolytic febrile transfusion reaction prevention; infection by cytomegalovirus; cytokine release reduction and others.

It has therefore become common, and indeed is a practice recommended and prescribed by regulatory bodies, to use filters for leukodepletion in preparing blood components.

With particular reference to these products, currently available filters comprise a casing, generally of plastic material, presenting an inlet port and an outlet port both connected to tubes through which the blood entering and leaving the casing flows. This casing contains a usually layered filter element consisting generally of mesh, membranes or more typically non-woven fabrics. The polymers most commonly used for the non-woven fabrics are polyesters, specifically PBT (polybutyleneterephthalate). U.S. Pat. No. 4,701,267 describes a filter unit for removing leukocytes from a suspension containing them, and in particular describes a filter element using a non-woven fabric with fibres of particular dimensions and density. This patent describes the materials specifically used for producing the constituent fibres of the non-woven fabric. EP329303 describes a method and device for leukodepletion of a platelet concentrate deriving from blood; the text describes various filter means obtained in PTFE (polytetrafluoroethylene), in PBT or in other fibres with hydroxyl or anionic groups. In particular, the use of polyester, polyamide and acrylic fibres is described.

The filter material is usually obtained by aggregating filtering layers which differ by physical characteristics, such as air permeability, average pore dimension, average fibre dimension and surface tension. Filtration results in leukocyte removal both by physico-mechanical action (screening) related to the cell dimensions (screen filtration), by the capacity of the leukocytes to adhere to the synthetic fibres (depth filtration) independently of their dimensions, and by the effect of biological phenomena related to the presence of protein molecules having adhesive properties present on the external surface of their cell membranes, (intergrine, selectine, immunoglobulin-like). The surface tension of the filter material is of extreme importance in determining the hydraulic strength of the filter and in promoting or not promoting the adsorption mechanism which governs the effectiveness of removing the particular substance (leukocytes) from the blood.

In this respect, it is known that high material surface tension reduces the platelet adhesion phenomenon during blood filtration. In contrast, low surface tension of the filter material gives rise to a hydrophobic interaction with the filtered liquid, with a greater pressure drop across the filter (less throughput for equal pressure difference) and a lack of permeation uniformity, with relative formation of channels via preferential circuits.

Filters of the aforestated types and methods for their production are known. For example, EP1897571 describes a filter for removing substances from blood or blood derivatives comprising a filtering polymer material having a polyurethane coating, wherein this latter has a number average molecular weight not exceeding 10,000 Dalton (Da). This polyurethane is obtained by an addition reaction of an aliphatic diisocyanate, a polyethylene glycol and butanediol, using an excess of this latter, so that the addition reaction is stoichiometrically unbalanced.

This known filter is obtained by a method involving the use of pollutant chemical products requiring particular attention for their disposal. This results in an increase in production costs and hence in the cost of the final product obtained.

Another patent text, WO2005/113136, describes a polyurethane material for leukocyte adsorption having a weight average molecular weight equal to or exceeding 20,000 Daltons; this prior text states that the use of a polyurethane of weight average molecular weight less than 20,000 Daltons is not adequate as this molecular weight is too low for a material which is to be used for leukodepletion.

Both these prior patents hence describe leukodepletion filters using filter elements having at least one layer coated with polyurethane. However this coating can present either a molecular weight which may be insufficient to adequately filter the blood, or on the contrary a molecular weight which is adequate, according to that stated by the inventors, but the application of which to the non-woven fabric uses methods involving the use of chemical solvents with consequent high risks to the environment. Hence for their implementation, these methods require means to prevent release of pollutant products into the atmosphere, these means representing a certain cost which is transferred to the finished product.

### SUMMARY OF THE INVENTION

An object of the present invention is to provide a filter able to remove substances from the blood, and a method for its formation, which represent an improvement over similar already known filters and methods.

A particular object of the invention is to provide a filter having high leukocyte retention and high platelet retention, with extremely low undesired release and optimal filtration times, and obtainable by a method which respects the environment and is of low environmental impact.

Another object is to provide a method of the stated type which is of low implementation costs and high efficiency.

These and other objects which will be apparent to the expert of the art are attained by a filter and method for its formation in accordance with the accompanying claims.

### DETAILED DESCRIPTION OF THE PREFERRED EMBODIMENTS

According to the invention, the filter is made of non-woven fabric consisting of a layer (or hydrophobic substrate) of PBT having low surface tension and high hydrophobicity. This filter comprises at least one layer of this material.

Said layer is coated with polyurethane material of which the number average molecular weight is between 10,000 and 20,000 Daltons (Da), for example between 10,000 and 19,000 Da, advantageously between 12,000 Da and 18,000 Da, and preferably between 15,000 Da and 17,500 Da.

In this respect it has been surprisingly found that with polyurethane of molecular weight within the aforesaid ranges, a filter can be formed for removing leukocytes and platelets from the blood. In particular, with these values it has been shown possible to reduce the total number of leukocytes by a value of the order of 10⁴-10⁶ times.

The any of the above-listed number average MW ranges may be further defined with a respective weight average MW proviso. For example, a proviso that the polyurethane does not have a weight average molecular weight of greater than or equal to 20,000 Daltons; or a proviso that the polyurethane does not have a weight average molecular weight of greater than or equal to 19,000 Daltons. Advantageously a proviso that the polyurethane does not have a weight average molecular weight of greater than or equal to 18,000 Daltons, and preferably a proviso that the polyurethane does not have a weight average molecular weight of greater than or equal to 17,500 Daltons.

The polyurethane coating is applied to the hydrophobic substrate by an impregnation technique, by immersing this latter in a container in which a mixture is present containing the polyurethane in a controlled concentration. The mixture consists of a solution in which the polyurethane is dissolved in an inert polar organic solvent, such as isopropanol or propylene glycol methyl ether, or preferably a dispersion in water. The polyurethane concentration in said mixture is from 0.5% to 20% by weight, preferably around 8%, whether in organic solution or in aqueous dispersion.

Alternatively, solutions or suspensions in mixtures of inert polar organic solvent and water can be used.

The following are used as polyurethane components: an aliphatic isocyanate (isocyanate —N═C═O group) with characteristics similar to diphenylmethane diisocyanate (MDI); a polyol (typically a polyethylene glycol or polyesters) with which the isocyanate forms a urethane bond.

The polyurethanes are produced by reacting an isocyanate containing two or more isocyanates groups per molecule (R—(N═C═O)_{n≧2}) with a polyol containing on average two or more hydroxy groups per molecule (R′—(OH)_{n≧2}), in the presence of a catalyst. Generally R is an aliphatic moiety.

Typical diisocyanate, which is used for the preparation of the polyurethane, is preferably an aliphatic diisocyanate, such as particularly isophorone diisocyanate (IPDI), hexamethylene diisocyanate (HDI), dicycloexyl methane diisocyanate (HMDI), trimethylhexamethylene diisocyanate, 1,3-bis(isocyanomethyl)cycloexane and diisocyanates of dimer acids.

The polymer diol is typically any one or more of polyethylene glycol, polypropylene glycol, polytetramethylene glycol, poly(ethylene glycol/propylene glycol) copolymer, poly(ethylene glycol/tetramethylene glycol) copolymer.

The impregnation technique is conventional and consists of immersing the non-woven fabric in a tank containing the mixture. The effect of the polyurethane coating on the non-woven fabric layer is that the surface tension of this latter increases. The layer hence loses the hydrophobicity characteristic and becomes hydrophilic and easily wettable with water. In addition the coating of the non-woven fabric enables the average pore diameter of the filtering layer to be controlled such that it is between 3 and 15 micron, hence enabling specific filtration characteristics to be obtained as a function of the average diameter of the blood components to be removed by mechanical filtration. For example, the average leukocyte diameter is between 4 and 10 micron, the average erythrocyte diameter being between 7 and 8 as reported in and known from the literature.

The non-woven fabric layer coated with polyurethane can be sandwiched between hydrophobic layers of known type (or rather between layers of materials less hydrophilic than the non-woven fabric layer coated with polyurethane).

Because of the particular method of forming the filter, this can be obtained in a manner such as not to be dangerous and polluting to the environment.

This enables special costly control instruments for the impregnation process to be avoided, as can those disposal costs for the solvents or products used in the state of the art for the currently known impregnation of the filtering layer, for example of PBT. This cost saving is evidently reflected in the finished product costs.

The invention is further illustrated by the following examples of a filter according to the invention, given by way of non-limiting example.

From the said examples it was found that by using an aqueous dispersion of polyurethane instead of a solution in organic solvent, filters could be obtained having greater coating stability, demonstrated by the smaller values of the residue in water compared with similar values obtained from a solution in organic solvent.

### Example 1

A dispersion of polyurethane of number average molecular weight between 12 and 18 kDa in water was used, with a polymer content of 5% by weight. This dispersion was obtained by the following process: the polyol and isocyanate components are dissolved in a ketonic solvent, then dispersed in water; the solvent is finally removed by distillation.

The polyurethane coating was applied to a non-woven fabric of PBT by immersion in the dispersion maintained at ambient temperature. Drying was carried out at 60° C. After 20 hours of drying, it was washed in osmotized water at 50° C. Subsequent drying was carried out at 60° C.

### Example 2

A dispersion of polyurethane of number average molecular weight between 12 and 18 kDa in water was used, obtained by diluting at ambient temperature a polyurethane dispersion in water, with a PU content of 50% by weight and with the further addition of water to attain a polymer content of 0.5% by weight.

The polyurethane was NORETHANE WB 8301 produced by the firm NOVOTEX ITALIANA SPA.

The polyurethane coating was applied to a non-woven fabric of PBT by immersion in the dispersion maintained at ambient temperature. Drying was carried out at 50° C. After 20 hours of drying, it was washed in osmotized water at 45° C. Subsequent drying was carried out at 30° C.

### Example 3

A dispersion of polyurethane of molecular weight between 12 and 18 kDa in water was used, with a polymer content of 5% by weight.

The polyurethane coating was applied to a non-woven fabric of PBT by immersion.

Drying was carried out at 100° C.

### Example 4

Instead of a polyurethane dispersion in water, a solution of polyurethane of molecular weight between 12 and 18 kDa in isopropanol was used, with a polymer content of 3% by weight.

The polyurethane coating was applied to a non-woven fabric of PBT by immersion.

These examples are summarized in the following TABLE I:

TABLE I

| Example | PU Base | Polyurethane concentration | |
|---|---|---|---|
| Example 1 | Water | 8% | PU |
| Example 2 | Water | 0.5% | PU |
| Example 3 | Water | 15% | PU |
| Example 4 | Organic | 3% | PU |
| | solvent | | |

Effectiveness of Leukocyte Retention, Platelet Retention, and Filtration Times

To compare filtration effectiveness, filters were produced suitable for large scale filtration of erythrocyte concentrates (laboratory filtration for blood banks). The filters were formed with a rigid plastic casing, each filter comprising 36 stacked layers each of 46 cm² surface, of the filtering material obtained in the stated examples. Bags of erythrocyte concentrates obtained by centrifugal separation from whole blood were filtered. The concentrates were used within the first 10 days from collection and were preserved at 4° C. before filtration.

The results of the tests carried out were compared, as shown in TABLE 2, with those obtained with filters produced in accordance with EP1897571 and WO2005113136, the data reported in these latter being utilized. In TABLE 2, Mn represents number average molecular weight and Mw represents weight average molecular weight.

The leukocyte and platelet count were obtained by an automatic cell count before and after filtration. The post-filtration leukocyte count was determined by a count in a Nageotte chamber.

TABLE 2
Comparison of GVS filtrations of the present invention with
Fresenius patent application no. EP 1897571A1 and University
of Southampton patent application no. WO 2005/113136A1

| | | | | U. |
|---|---|---|---|---|
| | | GVS | Fresenius | Southampton |
| Patent Application | | Present | EP | WO |
| | | specification | 1897571 A1 | 2005/113136A1 |
| Reference Example | | Present | Example 2 | Example 1 |
| in Patent App. | | Example 1 | | |
| Characteristics of | | 15,000 Da < | Mn < | 20,000 < Mw < |
| the PU used (Da) | | Mn < 17,500 | 10,000 Da | 1,000,000 Da |
| Type of blood used | | Red Blood | Whole Blood | Whole blood |
| for functional tests | | Cell | | |
| | | Concentrates | | |
| Size of samples | | Not | Discs 3 cm in | Discs 2 cm in |
| used - small scale | | available | diameter; 3 | diameter; 9 |
| | | | layers per | layers per filter; |
| | | | filter; 2 ml | 4 ml of whole |
| | | | of blood | blood filtered |
| Results | WBC | | 92% | 99.945% |
| | removal % | | | |
| | PLT | | 35% | 0% |
| | removal % | | | |
| Size of samples | | 36 layers | 40 layers | Not |
| used - large scale | | surface 46 cm² | surface 50 cm² | available |
| Results | Filtration | 17 | 14 | |
| | time (min) | | | |
| | PLT | 95% | 100% | |
| | removal % | | | |
| | Residual | 0.11 | 0.19 | |
| | WBC/ | | | |
| | unit × 10⁶ | | | |

The comparison with the University of Southampton solution is possible only with small scale samples, also present in EP1897571.

As can be seen from the table, the effectiveness of leukocyte (WBC) removal is much higher than that of EP1897571.

Evaluation of the Residue in Water

The water soluble extracts were evaluated for each of the filters prepared in the above examples. With reference to the standard ISO 1135-4, Chapter 6, Chemical Requirements, three filters were connected in series with a peristaltic pump and water was recirculated for 2 hours at a flow rate of 1 l/h and a temperature of 37° C. Samples of permeated liquid were collected at the end of the test and sample absorbance was analyzed within the range 250-320 nm.

The results of the measurements are summarized in TABLE 3. The WBC log reduction value was calculated by the formula:

Log₁₀ (leukocyte number prefiltration/leukocyte number postfiltration).

The platelet depletion value was calculated as:

platelet depletion=[(platelet number postfiltration/μl)/(platelet number prefiltration/μl)]%.

TABLE 3

| | Treatment | WBC Log | Filtration | Platelet | Absorb- |
|---|---|---|---|---|---|
| Ex. | parameters | reduction | time | depletion | ance |
| Ex 1 | 8% PU in | 7.0 | 19.2 | 95% | <0.2 |
| | water base | | | | |
| Ex 2 | 0.5% PU in | 3.4 | 5.7 | 100% | <0.2 |
| | water base | | | | |
| Ex 3 | 15% PU in | 2.0 | 13.5 | 50% | <0.2 |
| | water base | | | | |
| Ex 4 | 3% PU in | 4.0 | 14.2 | 90% | >0.2 |
| | organic | | | | |
| | solvent base | | | | |

It should be apparent that embodiments other than those expressly disclosed herein are also within the spirit and scope of the present invention and that the present invention is not limited by the above disclosure but rather is defined by the claims appended hereto.

1. A filter for removing substances from blood or from blood derivatives, said filter comprising a casing containing a layered filter element comprising a plurality of layers, at least one layer of this latter being coated with polyurethane, wherein said polyurethane has a number average molecular weight between 10,000 Dalton and 20,000 Dalton.

2. A filter as claimed in claim 1, wherein the polyurethane number average molecular weight is between 12,000 Dalton and 18,000 Dalton.

3. A filter as claimed in claim 1, wherein the polyurethane number average molecular weight is between 15,000 Dalton and 17,500 Dalton.

4. A filter as claimed in claim 1, wherein the polyurethane is obtained at least from an aliphatic isocyanate and a polyol with which the isocyanate forms a urethane bond.

5. A filter as claimed in claim 1, wherein the polyurethane is obtained at least from an aliphatic isocyanate (isocyanate —N═C═O group) with characteristics similar to diphenylmethane diisocyanate (MDI); and a polyol selected from the group consisting of a polyethylene glycol and polyesters with which the isocyanate forms a urethane bond.

6. A filter as claimed in claim 1, wherein the polyurethane coated layer is of non-woven fabric.

7. A filter as claimed in claim 1, wherein the polyurethane coated layer is of non-woven polymer fabric.

8. A filter as claimed in claim 1, wherein the polyurethane coated layer is of non-woven fabric comprising polybutyleneterephthalate or polyester.

9. A filter as claimed in claim 1, wheren the polyurethane coated layer is sandwiched between less hydrophilic layers.

10. A method for forming a layered filter element for a filter for removing substances from blood or from blood derivatives, said filter element comprising at least one layer of non-woven fabric which is impregnated with polyurethane by immersing it in a container or tank of a mixture in which polyurethane is present, wherein this mixture consists of a solution in which the polyurethane is dissolved in a polar solvent or dispersed in a mixture containing a polar solvent.

11. A method as claimed in claim 10, wherein the polar solvent is water.

12. A method as claimed in claim 10, wherein the polar solvent is an inert polar organic solvent such as isopropanol or propylene glycol methyl ether.

13. A method as claimed in claim 10, wherein the mixture is a mixture of an inert polar organic solvent and water.

14. A method as claimed in claim 10, wherein the polyurethane concentration in the mixture is from 0.5% to 20.

15. A method as claimed in claim 10, comprising:
immersing the layer to be coated into the tank containing the mixture,
leaving this layer immersed in the mixture for a time between 10 and 30 seconds,
extracting the layer from the tank and subjecting it to squeezing,
drying said layer in an oven for a time of 5-20 minutes, said oven being at a temperature between 30° C. and 110° C.,
then after drying washing the dried layer at a temperature between 40° C. and 50° C.,
followed by further drying at a temperature between 30° C. and 60° C.

16. A method as claimed in claim 10, comprising controlling the pore diameter of the non-woven fabric through the polyurethane coating, said diameter being between 3 and 15 μm.

17. A method as claimed in claim 10, wherein the layered filter element is for a filter for removing substances selected from at least one member of the group consisting of leukocytes and platelets from blood or from blood derivatives.

18. A method as claimed in claim 15, wherein said oven being at a temperature between 45° C. and 55° C. and said washing comprises washing the dried layer with osmotized water at the temperature between 40° C. and 50° C.

19. A method as claimed in claim 10, wherein the polyurethane concentration in the mixture around 6-10% by weight.

20. A method as claimed in claim 10, wherein the polyurethane concentration in the mixture around 8% by weight.
